(19) United States
(12) Patent Application Publication
(10) Pub. No.: US 2008/0125835 A1
(43) Pub. Date: May 29, 2008

(54) METHOD AND APPARATUS FOR LIGHT THERAPY

(76) Inventor: Richard Laurent, Bloomfield Hills, MI (US)

Correspondence Address: ROBERT M. SCHWARTZ, P.A., P.O. BOX 221470, HOLLYWOOD, FL, 33022, US

(21) Appl. No.: 11/771,526

(22) Filed: Jun. 29, 2007

Related U.S. Application Data
(63) Continuation-in-part of application No. 11/701,600, filed on Feb. 2, 2007, now abandoned.
(60) Provisional application No. 60/764,471, filed on Feb. 2, 2006.

(51) Int. Cl. A61B 18/20

(52) U.S. Cl. 607/89

(57) ABSTRACT

A laser device for stimulating a body region is disclosed. The laser device includes a housing structure having a head assembly, a plurality of pins, a plurality of laser modules, and a microcontroller. The pins are configured to stimulate a portion of a body region, without affecting the target area of the lasers. The laser modules are configured to provide laser energy to the body region.

### CROSS REFERENCE TO RELATED APPLICATIONS

This application is a continuation-in-part of non-provisional application Ser. No. 11/701,600, entitled A METHOD AND APPARATUS FOR STIMULATING HAIR GROWTH, filed on Feb. 2, 2007, which claims priority to provisional application 60/764,471, entitled A METHOD AND APPARATUS FOR STIMULATING HAIR GROWTH filed Feb. 2, 2006, the entire contents of which applications are hereby incorporated herein by reference in their entirety.

### BACKGROUND

There are many known methods and uses for providing and promoting the regrowth of hair, treatments for acne and skin blemish control, treatments for periodontal diseases and hypersensitivity, wound epitelization (healing) and cutaneous aging (anti-aging). Some of these therapies require the use of topical creams, surgical implants, laser augmentation, medications, etc. for treating thinning hair or alopecia (human hair loss), injection of toxins for treatment of wrinkles (anti-aging), or to attenuate pain and reduce inflammation associated with arthritis, etc. Many of these treatments are of limited usefulness, are invasive, use medication, are complicated and/or expensive. What is needed therefore is an effective, affordable and drug-free alternative to treat, prevent or impede many disorders.

### BRIEF DESCRIPTION OF THE DRAWINGS

FIG. 1A illustrates a front view of a handheld laser device according to an exemplary approach;

FIG. 1B illustrates a side view of the laser device according to FIG. 1A;

FIG. 2A illustrates a side view of a laser device according to an exemplary approach, positioned against a treatment area;

FIG. 2B illustrates a partial exploded view illustrating treatment using the laser device of FIG. 2A;

FIG. 3A illustrates an isometric view of a laser device 100″ according to an embodiment;

FIG. 3B illustrates a front view of a handheld laser device of FIG. 3A;

FIG. 3C illustrates a side view of a handheld laser device of FIG. 3B;

FIG. 4A illustrates a side internal view of an exemplary laser device;

FIG. 4B illustrates a side internal view of a modular head assembly;

FIG. 5A illustrates a front view of a laser device according to an exemplary approach;

FIG. 5B illustrates a partial exploded view of the laser device of FIG. 5A;

FIG. 6A illustrates an isometric view of a laser module and associated mounting hardware according to an embodiment;

FIG. 6B illustrates an exploded view of the laser module and associated mounting hardware of FIG. 6A;

FIG. 7 is a flow chart illustrating the functionality of a laser device according to an exemplary approach; and

FIG. 8 illustrates an exemplary schematic of a laser device according to an exemplary approach;

### DETAILED DESCRIPTION OF THE INVENTION

A method and apparatus for providing Low Level Laser Therapy (LLLT) is provided.

The basic principle behind LLLT is that coherent light emissions can affect and improve cellular dynamics. One method in which this can occur is through cellular energization, which transforms laser energy into cellular energy (e.g. photons to ions). Laser photons may increase the energy, including ion energy, available to cells so they take in nutrients and chemical fortifications faster, and dispose of indigenous waste bi-products more readily. Further higher rates of ATP, RNA and DNA are synthesized and increased levels of circulation, increasing blood arterial, venous and lymph micro circulation through vasodilatation. Laser light energy may also increase stimulatory and regulatory mechanisms of the skin cellular metabolism, blood circulation and oxygenation of the skin and scalp tissues, thereby helping to carry more essential minerals and nutrients into the areas where exposure from this laser energy subsisted. As a result of this increased blood flow, toxins and indigenous cellular waste bi-products, including harmful dihydrotestosterone (DHT) may be taken away in a more expedient matter. Laser energy also removes calcification and blockages around cells, increases cell replacement, regenerative, and proliferation function activities.

The device includes a plurality of laser modules configured to stimulate a treatment area such as hair follicles, papilla, and surrounding tissue and cells by providing free space coherent light energy. Body regions may include, but are not limited to, the face, head, arms, legs, and chest. One common use for LLLT is for stimulating hair growth on the head, where the treatment region includes the hair, follicles, skin and scalp. The apparatus may further include a plurality of pins, selectively positioned with respect to one or more laser modules, which pins may be in contact with the treatment region during use of the device.

In one exemplary approach, the device may be a hand-held, battery operated device shaped similar to a hairbrush. In another exemplary approach, the device may be a hood-shaped device, configured to be placed over a treatment area, such as the head of a user, similar to a commercial hair dryer. A hood-shaped device may be configured, e.g., to be mounted on a wall, or on a floor-standing pedestal.

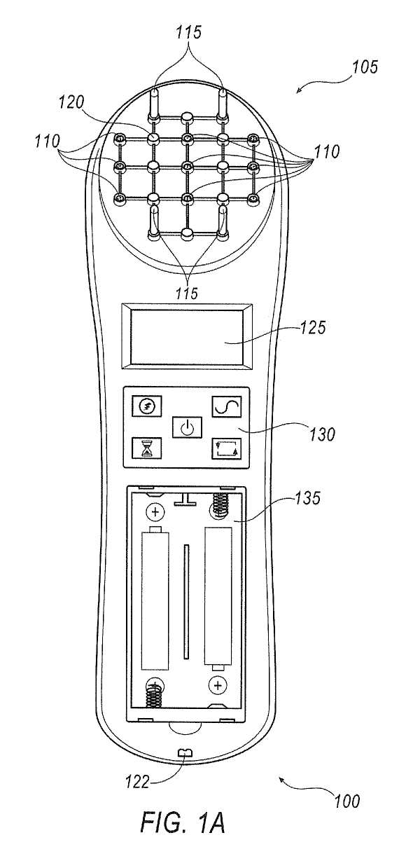

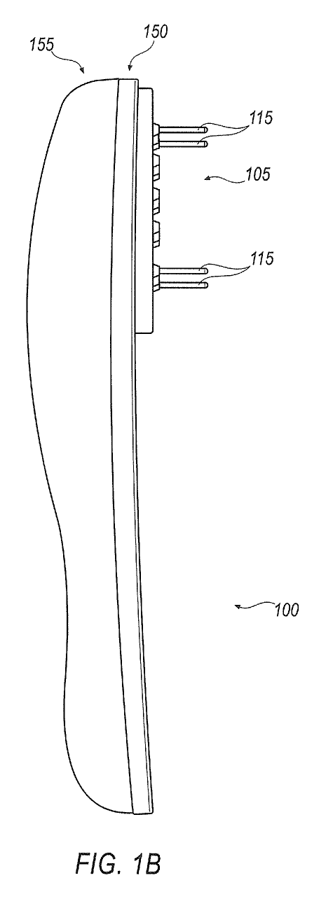

FIG. 1A illustrates an exemplary handheld laser device 100 according to an exemplary approach. Laser device 100 comprises a head assembly 105, a data port 122, an LCD display 125, a control pad 130, and a battery compartment 135. The housing of the laser device 100, as best shown in FIG. 1B, includes an upper housing portion 150 and a lower housing portion 155. In the illustrated approach, the head assembly 105, LCD display 125, control pad 130 and battery compartment 135 collectively represent the upper housing portion 150. The lower housing portion 155 serves primarily as a cover, to seal and protect the internal components of the laser device 100. In another exemplary approach, one or more of the data port 122, LCD display 125, control pad 130, and battery compartment 135 may be disposed within the lower housing portion 155. The housing is generally made of plastic, though other materials may be suitable as well.

Referring to FIG. 1A, a plurality of holes 120 are defined within a face of head assembly 105. The plurality of holes 120 may be configured, e.g., as a mounting location for laser modules 110, pins 115, sensors (not shown), laser diodes (not shown), transducers (not shown), etc. In the exemplary approach of FIG. 1A, four pins 115 are selectively positioned with respect to five lasers 110. However, this is by way of example only, and not of limitation, and it will be understood that any number of pin 115 and laser 110 combinations are contemplated herein.

Laser modules 110 may include a laser diode, a photo diode, a power control circuit, a lens and a housing. The laser diode may be a tunable laser diode, capable of providing laser energy in one or more wavelengths and/or intensities. The lens may be configured to, e.g., collimate a beam, focus a beam, expand a beam, etc.

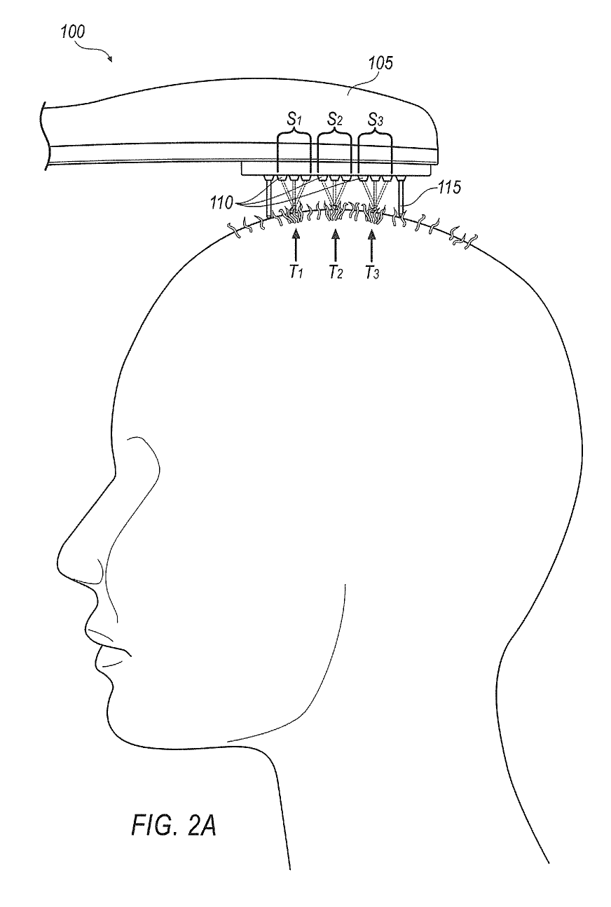

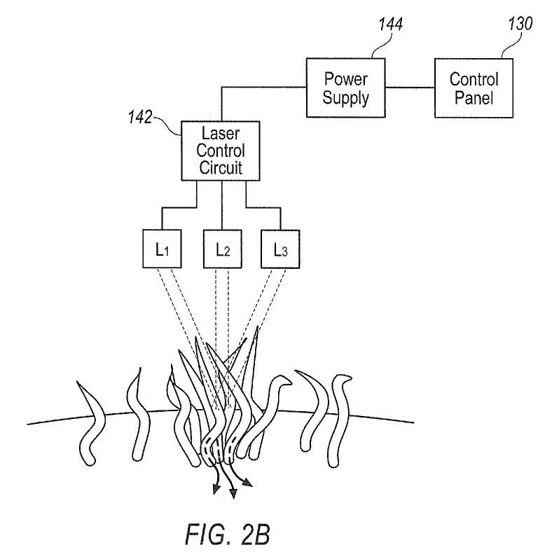

Laser modules 110 may be mounted within the head assembly 105. The orientation of laser modules 110 within head assembly 105 may depend in part on the number of laser modules 110, pins 115, motors, sensors and transducers that are installed in the head assembly 105, as well as the intended LLLT treatment to be provided. In one exemplary approach, laser modules 110 may be mounted such that a subset of laser modules 110 may project focused beams to contact a target area at substantially the same location, as best illustrated in FIG. 2A.

In the illustrated approach of FIG. 2A, the handheld device 100 includes nine laser modules 110. The modules 110 are positioned within the head assembly 105 such that focused beams projected by the laser modules 110 impinge on one of three distinct treatment regions T1, T2, T3. In the illustrated approach, the nine modules 110 comprise three subsets S1, S2, S3, each subset having three laser modules 110. The three laser modules 110 in the first subset S1 may be angled such that the three laser modules 110 are all focused on a first treatment region T1, with the beam of each laser module 110 of subset S1 substantially overlapping at a first focal point. The three laser modules 110 in the second subset S2 may be similarly angled such that the three laser modules 110 are all focused on a second treatment region T2, with the beam of each laser module 110 of subset S2 substantially overlapping at a second focal point. Finally, the three laser modules 110 of the third subset S3 may be similarly angled such that the three laser modules 110 are all focused on a third treatment region T3, with the beam of each laser module 110 of subset S3 substantially overlapping at a third focal point. By so positioning the laser modules 110, a given treatment region T1, T2, T3 may receive an increased amount of energy in comparison to an area exposed to a beam from a single laser module 110. While the beams from laser modules 110 within a given subset S1, S2, S3 are described as substantially overlapping and/or converging, it is to be understood that this is by way of example only, and not of limitation. One of skill in the art recognizes that similar results can be obtained by beams from multiple laser modules 110 impinging and/or converging in a substantially similar area, such as within the same square centimeter of treatment area.

FIG. 2B illustrates a partial exploded view of an exemplary approach such as that illustrated in FIG. 2A. In the illustrated embodiment, a control panel 130 is used to select, e.g., a pulsing scheme, an optical power intensity scheme, and treatment duration for a plurality of lasers L1, L2, L3, which may be laser modules 110. Control panel 130 may also be used to select, e.g., activation of device options, such as pin vibration and activation, temperature, sensory measurements, etc. A power supply 144, such as a battery, provides power to a laser control circuit 142 and to the plurality of lasers L1, L2, L3. The laser control circuit 142 is configured to pulse lasers according to a chosen pulse interval chosen by a user through the control panel 130. The laser control circuit 142 may also be configured to control the power output of lasers according to a power intensity chosen by a user through the control panel 130. The laser modules L1, L2, L3 are positioned such that the beams from the respective lasers L1, L2, L3 are focused on a common focal point, as described previously with respect to FIG. 2A. As the beams travel from the laser modules L1, L2, L3 to the convergence or focal point, they generally may impinge on tissue and/or on one or more hairs in the treatment area. When a beam encounters a hair, the beam may enter the shaft of the hair. The temperature of the hair may increase, due to melanin (chromophore) absorption of this light energy. The hair then acts as a filament conductor, similar to a fiber optic, transmitting this energy along the length of the hair shaft, to the hair follicle. The energy may be further transmitted within the follicle to the root and/or the dermal papilla (root) of the hair, the scalp and the surrounding tissue. This may further energize the treatment area and facilitate hair cell regeneration and/or nutritional fortification. As multiple beams may impinge on a single hair, or on a common point within a treatment area, such as a tissue area, the energy supplied to a treatment area may be substantially more than the energy provided by a single laser module 110, and the energy from a first laser module 110 may reinforce the energy from a second laser module 110. Accordingly, improved treatment may be provided to a user, and lower power laser modules 110 may be utilized, without sacrificing the efficacy of the photobiostimulation. Using lower powered laser modules 110 may provide advantages in terms of cost, energy consumption, and safety to a user, without sacrificing treatment quality.

Laser control circuit 142 may pulse beams from lasers L1, L2, L3 according to one or more pulsing modes. Pulsing lasers L1, L2, L3 provides intermittent beams to the target area, which allows cells, tissues, hair papilla, follicles and hair to rest and/or resonate. Allowing a target area to rest and/or resonate may increase the effects of cellular stimulation, and may thereby aid and promote cellular proliferation and regeneration, hair growth, collagen development, etc. Accordingly, LLLT may be made more effective by pulsing laser beams, causing a target area to rest and/or resonate. Pulsing modes may indicate, e.g., the frequency of pulses, dictating the number of pulses provided per second, the power and duty cycle of the pulse, indicating the percentage of time the laser is powered between pulses, the power characteristics of the pulse, such as whether the pulse is fully powered during the entire “on” segment of a pulse or whether power ramps up or down to a particular power level, etc.

Pulsing beams from lasers L1, L2, L3 may reduce power consumption, such as by reducing laser diode 110 thermal runaway, which may extend battery life of a laser device 100, by providing power to lasers L1, L2, L3 intermittently. Further, intermittently powering off lasers L1, L2, L3, or periodically reducing laser power intensity, may also extend the useful life of the device, the laser modules L1, L2, L3, etc.

Pulsing a laser may be accomplished by altering the laser duty cycle. For instance, in an exemplary embodiment, as opposed to having constant power provided to a laser module, the laser module may be provided with a 20% duty cycle, where a laser module is powered on for a first time period (the “on-cycle”), followed by a second time period, four times the length of the first, in which the laser is not powered (the “off-cycle”). The cycle may then be repeated. Subsequent cycles may use the same duty cycle, or may use alternate duty cycles, from 0 to 100%.

In addition, pulsing may further be configured to provide additional control over power levels. For instance, in the preceding exemplary embodiments, a duty cycle comprises a first period (the “on-cycle”) where the laser is powered, followed by a second period (the “off-cycle”) where the laser is not powered. Additional embodiments may be provided wherein the on-cycle of the duty cycle may comprise sub-duty cycles, wherein power is pulsed during the “on-cycle” according to one or more duty cycles.

Pins 115 in head assembly 105 may serve as a mechanism to regulate distance between laser modules 110 and a body region being treated. By maintaining the distance between the emanating aperture of the lasers 110 and the body region subjected to treatment, the optimum amount of laser energy is appropriately distributed without maximizing the intensity of the laser energy and over-exposing the body region exposed to treatment. In addition to regulating power intensity through pins 115, power intensity may also be affected by the number of laser modules 110 providing laser energy to a given treatment area, the power output of the laser modules 110, the pulsing scheme of the laser modules (e.g. the duty cycle, etc.), the lens through which laser energy travels, the surface area over which the energy is provided, etc.

Pins 115 may be retractable and/or compression spring type pins, allowing the pins 115 to conform to the changing contours of a treatment area, and to remain in constant contact therewith. Pins 115 may be mounted within head assembly 105, such as on an inner face proximate one or more holes 120 defined within the head assembly 105, on a printed circuit board 175 disposed within the head assembly 105, etc.

By selectively combining pins 115 and laser modules 110, a treatment region may be defined wherein laser energy is distributed to a substantially undisturbed portion of a body region being treated. More specifically, pins 115 and laser modules 110 may be mounted with respect to one another such that pins 115 and laser modules 110 act independently of one another, allowing laser modules 110 to impact a substantially undisturbed treatment region

Pins 115 in head assembly 105 may serve a number of purposes. As mentioned above, pins 115 may serve to regulate distance between apertures of laser modules 110 and a body region being treated. Furthermore, pins 115 may be configured, by way of example and not of limitation, for additional tissue stimulation, measurement devices, distance and/or positioning sensors, transducers and/or electrodes.

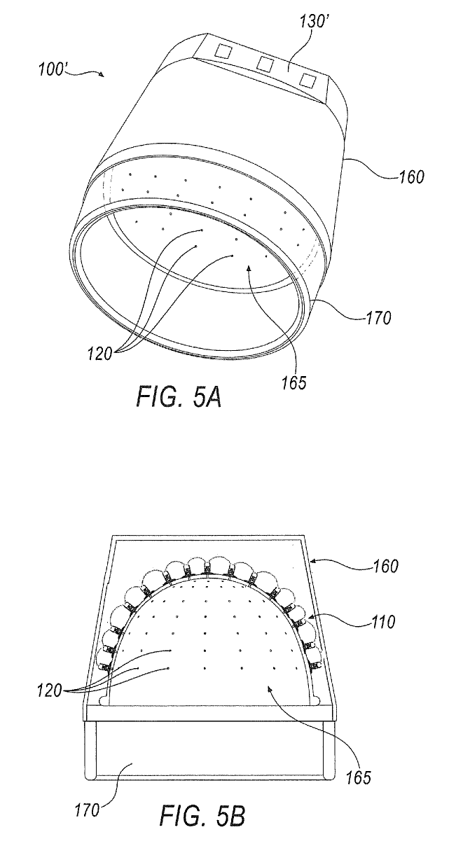

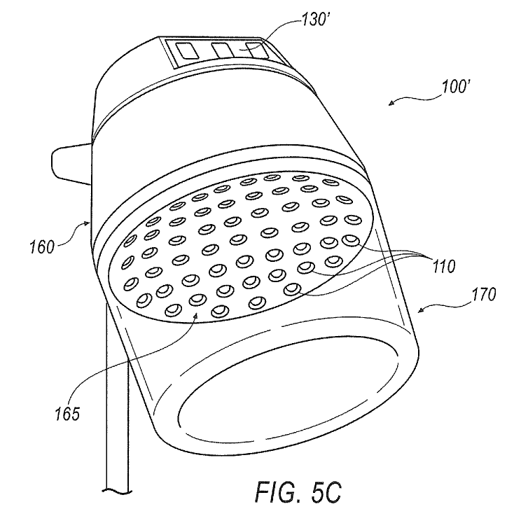

Configuring pins 115 for additional tissue stimulation may allow for increased blood circulation, and/or help free debris from treatment area, such as by freeing indigenous debris from follicles in the treatment area. In one exemplary approach, pins 115 may be operatively connected to one or more motors configured to vibrate the pins 115 to massage the tissue in a treatment area. The motors may be, e.g., vibrating, off-axis motors. In another exemplary approach, ring magnets 154 (FIG. 4B) may be disposed around pins 115. In an exemplary approach, wherein pins 115 are created from a magnetizable material, ring magnets 154 may magnetize pins 115 for further cellular and tissue stimulation of a treatment region, while attracting unwanted debris and contaminants. In a further exemplary approach, pins 115 may also act as electrodes, incorporated to induce a gentle electro pulse high voltage, low current to the treatment area, providing additional ionic and/or electrical stimulation.

One or more positioning sensors or switches may be operably connected to one or more pins 115 to determine when the device 100 is placed in contact with the desired treatment region, such as the scalp of a user. In addition, sensory devices, such as acoustic, sonic, ultrasonic, inductive, magnetic, optical, speed, proximity LED's, thermistors, thermal couples, RTD, optocouplers, optoisolators device, etc. may be incorporated or connected to the metal pins 115, or to springs mounted proximate to pins 115 to measure, e.g., resistance (ohmic), impedance, reactance, current, voltages, flow, pressure, thermal, light or chemical properties within a treatment area. Laser device 100 may use readings from positioning sensors or transducers devices attached to pins 115, as an indicator that the laser device 100 is positioned in a treatment region. The device 100 may use these readings to determine when to provide power to the one or more laser modules 110. By ensuring that device 100 is properly positioned prior to powering laser modules 110, or by removing power from laser modules 110 when laser device 100 is removed from a treatment area, power draw for the laser device 100 may be reduced, and radiation emissions from laser modules 110 will not be emitted to unintended areas

The head assembly 105 of laser device 100 may be modular and removable, allowing a user to easily interchange head assemblies 105. By interchanging head assemblies 105, a single laser device 100 may serve a series of different functions, and provide a series of different LLLT therapeutic applications, through the use of a variety of different lasers 110, pins 115, and combinations thereof. Laser modules 110 may be provided which are capable of operating at different wavelengths of light (WOL), different or variable power levels, and at varying pulse durations to accommodate varying treatment applications. Pins 115 may be provided which include any of a variety of optional functions, such as laser power activation, vibrating massage, magnetization, high-voltage electro-pulse, photon and tissue quantitative measurement capabilities, transducing and conducting temperatures, etc. Thus a user may use LLLT from device 100 to treat a myriad of conditions, including hair loss and rejuvenation, acne and blemish control, wrinkle reduction, skin rejuvenation (Anti-aging, cutaneous aging), anti-inflammation and arthritis control, wound epitelization (healing), hair reduction (epilation), periodontal disease and hypersensitivity treatment, muscle rejuvenation, sanitizing, sterilizing and tanning. By way of example, and not of limitation, to stimulate hair growth the head assembly 105 may provide laser energy with a wavelength typically within the range of 630-670 nanometers (nm). To treat skin for wrinkle reduction (e.g. anti-aging), a modular (i.e. interchangeable) head assembly 105 may provide laser energy with a wavelength generally in the range of 650-830 nm. Including pins 115 with a vibrating feature, a magnetizing feature, or a thermally conductive feature, may increase blood circulation in the treatment area, which may increase the effectiveness of the LLLT. Pin 115 vibration may be at an acoustic level, such as ultrasonic or infrasonic levels. Laser modules 110 within a head assembly module 105 may have a common specified wavelength range. Alternatively, laser modules 110 within a head assembly module 105 may not have a common specified wavelength range, but may include two or more specified wavelength ranges within the same head assembly 105. Further, laser modules 110 within a head assembly module 105 may include one or more tunable lasers, said tunable lasers capable of providing laser light in two or more selectable wavelengths. Head assemblies 105 may be provided with quick disconnect method, such as a connector 154 (FIG. 4A) allowing for quick replacement of head assemblies 105. An exemplary quick-disconnect method is described below, with respect to FIGS. 4A and 4B

Data port 122, as illustrated in FIG. 1A, may be configured, e.g., to allow information to be exchanged between a laser device 100 and, e.g., a computer. Data port 122 may include, e.g., a Unified Standard Bus (USB) port, a mini-USB port, etc. A user may use data port 122 to connect to a computer and change programmable treatment settings, such as laser energy dosages and durations to the treatment areas via pulsing modes, power intensities, time durations, etc. Additionally, a user may thus track device usage using a computer, and may thereby store historic treatment data, such as time durations, pulse durations, power levels, intensity (e.g. joules per square centimeter), temperature settings, sensor readings (e.g. tissue impedance, temperature readings, etc), number of treatment regiments, etc. Additionally, software or firmware upgrades for a laser device 100 may be uploaded or downloaded from a computer to a laser device 100 through such a data port 122. Data port 122 may also be used to recharge batteries within laser device 100.

Battery compartment 135, as illustrated in FIG. 1A, may be configured to house one or more batteries to power the laser device. The batteries may be of any appropriate design to provide the necessary power to laser device 100. In one exemplary approach, battery compartment 135 may be configured to house one or more disposable batteries. In another exemplary approach, battery compartment 135 may be configured to house one or more rechargeable batteries. In the latter exemplary approach wherein battery compartment 135 may house one or more rechargeable batteries, laser device 100 may further include a charging circuit (not shown). In one exemplary approach, such a charging circuit may be operatively connected to data port 122 to provide power from an external source to the charging circuit to recharge one or more housed batteries. In another exemplary approach, such a charging circuit may include one or more separate connectors configured to provide power from an external source.

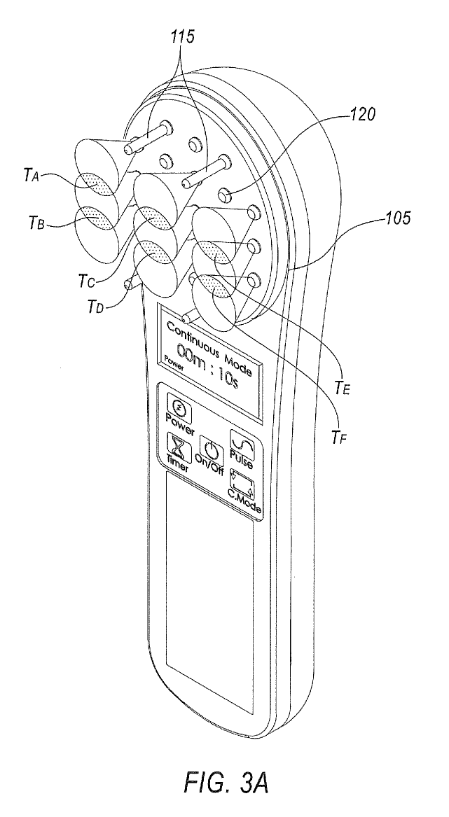

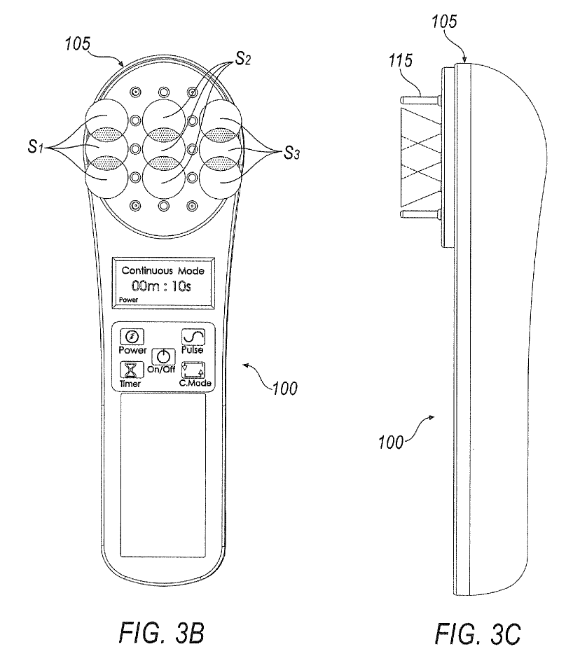

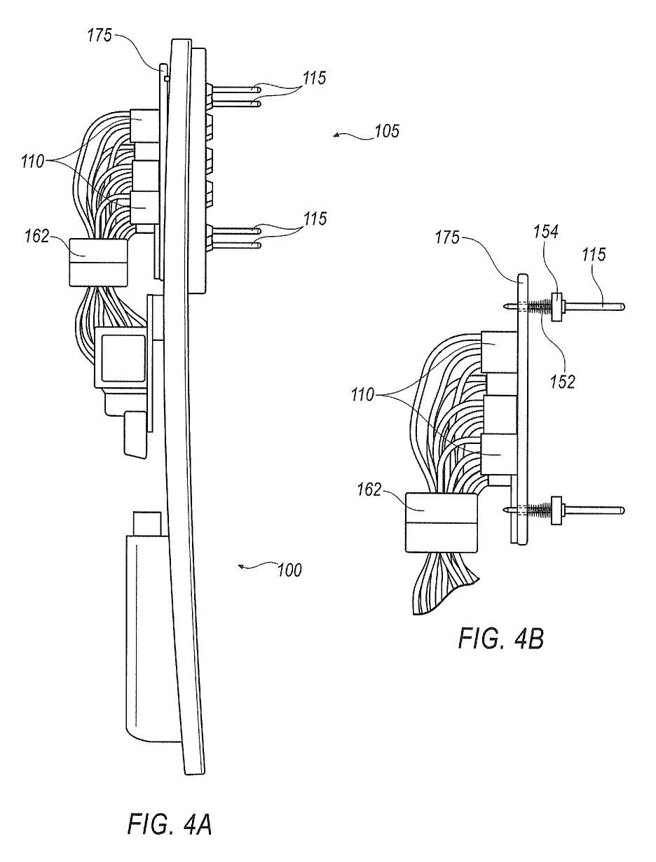

FIGS. 3A-3B illustrate a series of views of a handheld device 100 according to an embodiment. FIG. 3A illustrates an isometric view of the handheld device 100. The laser modules 110 in the illustrated embodiment project beams in a conical fashion. The nine laser modules 110 are divided into three subsets, S1, S2 and S3 (FIG. 3B). Some elements which are substantially the same as elements described with reference to FIGS. 1A and 1B may not be numbered, though this is for purposes of clarity of drawings only.

In the illustrated approach of FIG. 3A, the handheld device 100 includes nine laser modules disposed within a head assembly 105. The modules 110 are configured to provide a beam having a generally conical shape and are positioned within the head assembly 105 such that conical beams projected by the laser modules 110 overlap in one of six enhanced treatment regions T_{A}, T_{B}, T_{C}, T_{D}, T_{E}, T_{F}. In the illustrated approach, the nine modules 110 comprise three subsets S1, S2, S3, (FIG. 3B) each subset having three laser modules 110. The beams from the three laser modules 110 in the first subset S1 may be angled such that the three beams overlap and reinforce one another in two enhanced treatment regions, T_{A}, T_{B}, with the beam of a first laser module 110 and a second laser module 110 of subset S1 substantially overlapping at a first location T_{A}, and with the beam of the second laser module 110 and a third laser module 110 of subset S1 substantially overlapping at a second location T_{B}. The three laser modules 110 in the second subset S2 may be similarly angled such that the three beams overlap in two enhanced treatment regions, T_{C}, T_{D}, with the beam of a first laser module 110 and a second laser module 110 of subset S2 substantially overlapping at a first location T_{C}, and with the beam of the second laser module 110 and a third laser module 110 of subset S2 substantially overlapping at a second location T_{D}. Finally, the three laser modules 110 of the third subset S3 may be similarly angled such that the three beams overlap in two enhanced treatment regions, T_{E}, T_{F}, with the beam of a first laser module 110 and a second laser module 110 of subset S3 substantially overlapping at a first location T_{E}, and with the beam of the second laser module 110 and a third laser module 110 of subset S3 substantially overlapping at a second location T_{F}. By so positioning the laser modules 110, a given treatment region T_{A}, T_{B}, T_{C}, T_{D}, T_{E}, T_{F} may receive an increased amount of energy in comparison to an area exposed to a beam from a single laser module 110.

FIG. 3B illustrates a front view of the device 100 of FIG. 3A, further illustrating the overlap of conical beams of laser modules 110 from each of subsets S1, S2 and S3.

FIG. 3C illustrates a side view of the device 100 of FIG. 3A. Pins 115 may extend from a face of head assembly 105. The function of the pins 115 may include, by way of example and not of limitation, acting as a distancing mechanism to ensure that beams from modules 110 in subsets S1, S2 and S3 overlap an appropriate distance from a treatment region.

FIG. 4A illustrates an exemplary side view of a laser device 100, such as laser device 100 shown in FIGS. 1 and 2, illustrating the mounting configuration of the laser modules 110 in the head assembly 105. Laser modules 110, pins 115, motors, sensors and transducers may be mounted, e.g., on a printed circuit board (PCB) 175 platform. The PCB 175 assembly may be modular, and provided with a quick-disconnect method, such as connector 162, to allow for easy replacement of a PCB 175 with another. Connector 162 may include a first and a second connector portion. A first portion of connector 162 may be connected to a plurality of wires operably connected to, e.g., laser modules 110 mounted to PCB 175. A second portion of connector 162 may be connected to a plurality of wires operably connected to, e.g., a microcontroller, a power supply, etc. PCB 175 may be held in place, e.g., by bottom housing portion 155. Alternatively, PCB 175 may be held in place by one or more clips (not shown) disposed on upper housing portion 150. In this way, an entire head assembly 105 can be easily interchanged or removed by the user. In one exemplary approach, laser modules 110 may be mounted substantially perpendicular to the surface of the head assembly 105 such that the beams from laser modules are directed substantially downward. In another exemplary approach, laser modules 110 may be mounted in the holes 120 at predetermined angles to converge to effectively adjust the power provided to the area of the treatment region.

FIG. 4B illustrates a head assembly 105 such as that illustrated in FIG. 4A, according to an exemplary approach. Head assembly 105 includes a PCB platform 175 to which is mounted a plurality of laser modules 110 and a plurality of pins 115. Wires from the laser modules 110 are connected to a first end of a connector 162, allowing for quick connection/disconnection of the laser modules 110, and the head assembly 105. Pins 115 are configured to pass through PCB 175. Ring magnets 154 are disposed around a portion of respective pins 115. Springs 152 are disposed between pin 115 or ring magnet 154 and PCB 175, thereby allowing pins 115 to be retractable when depressed against a treatment area.

Functionality of laser modules 110 may be controlled by an internal microcontroller. The microcontroller may include a CPU core, LCD driver, SRAM, timers, programmable ROM, alarm generators, oscillator, timer/counters with pre-diver circuits and I/O ports. A microcontroller may be configured to be selectively programmed and may control, e.g., laser modules 110, motors connected to, or interacting with, pins 115, electro pulse voltage at different power intensities, laser modules 110 at different intensities, laser pulse intervals and time durations. For example a microcontroller may include a laser control circuit 142, as shown in FIG. 2B, configured to pulse laser modules 110 according to one or more preprogrammed pulsing schemes and/or power intensity levels. By programming pulse intervals and operating voltages, as well as power intensities, duty cycles, etc., of lasers modules 110, laser diode thermal runaway, current drain and hence the power dissipation from a power source may be reduced, thereby extending the battery life of the laser device 100, as well as the useful life of the laser modules 110, while still providing the desired stimulation and treatment. In this way, overall voltage and current consumption is reduced, thereby extending the battery life of the laser device 100, yet still maximizing laser energy for LLLT treatment in a pulsed mode.

A microcontroller may further include a memory configured to store, e.g., control software for operation of the laser device 100, data representative of laser device 100 usage such as laser dosage or power, number of individual treatment sessions, duration of individual treatment sessions, pulse durations and intervals, total laser device 100 treatment duration, etc. The microcontroller may transmit such information through data port 122 between the laser device 100 and, e.g., a computer. The microcontroller may further be configured to receive updated treatment data, such as updated pulsing modes, power levels, treatment duration, device 100 treatment options, etc., from e.g., a computer. Additionally, microcontroller may be configured to receive software upgrades for a laser device 100, which may be uploaded from a computer, through data port 122.

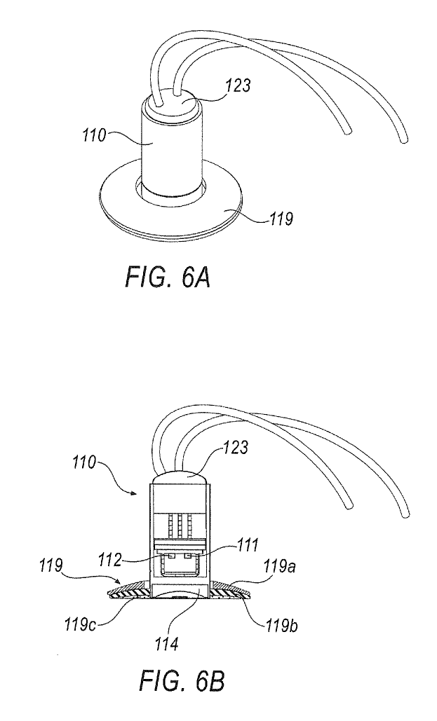

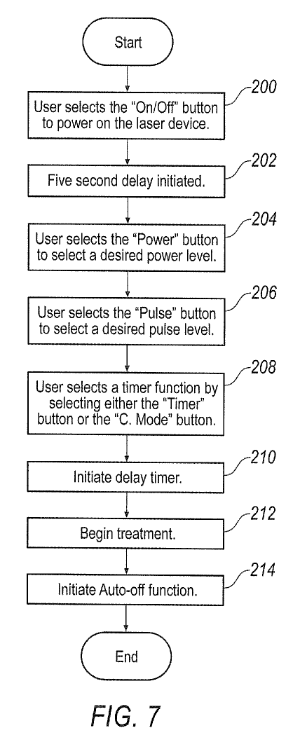

FIG. 5A illustrates an embodiment of a hood-style laser device 100′. Laser device 100′ comprises a housing 160 having a concave inner face 165. In one exemplary approach, housing 160 may have a hemispherical inner face 165. In another exemplary approach, housing 160 may have an ellipsoidal inner face 165. A plurality of laser modules 110 may be disposed within housing 160. Laser modules 110 may be positioned within housing 160 such that beams from laser modules 110 are directed through a plurality of corresponding holes 120 defined within the inner concave face 165 of housing 160. Laser device 100′ may further include one or more cooling mechanisms configured to cool a user of device 100′ and/or to cool the device 100′ itself, though the device 100′ may not require cooling due to the innovative pulsing mechanisms which may be employed.

The device 100′ of FIG. 5A may further include a control panel 130′ configured to allow control of the functionality of laser device 100′. The control panel 130′ may include LED indicators indicative of system status, such as a system mode, power status, pulse level, cooling system fan speed, etc. The control panel 130′ may further include an LCD display configured to provide information such as power level, and device 100′ setting information. Further, the control panel 130′ may include one or more controls configured to adjust one or more device 100′ settings.

Additionally, the device 100′ may include a transparent protective sheath 170 disposed around as least a portion of the housing 160. The protective sheath 170 may ensure that substantially all of the radiant energy from laser modules 110 is directed toward the treatment region, and is not directed to the area surrounding the device 100′.

FIG. 5B illustrates a partial exploded view of the device 100′ of FIG. 5A. A plurality of laser modules 110 are disposed within the housing 160 proximate a plurality of holes 120 defined within the inner concave face 165 of housing 160. In one exemplary illustration, inner concave face 165 may have an elliptical cross section. In another exemplary illustration, inner concave face 165 may have a semi-circular cross-section, or any other cross-sectional design, as appropriate. The geometrical cross-section of the inner concave face 165 may be determined by the nature of the focus energy pattern desired from the laser modules 110.

The device 100′ may be mounted, e.g., on a floor stand, or on an arm extending from a mounting surface such as a wall or a ceiling. The device 100′ may be mounted such that the position of laser device 100′ is adjustable, to allow for easier placement of laser device 100′ around a treatment area.

In one exemplary approach, a hood-style laser device 100′ includes one hundred laser modules 110 disposed within an ellipsoidal concave inner face 165 of housing 160. Housing 160 may be mounted to an adjustable arm which allows laser device 100′ to be placed around a treatment area, such as a user's head. Control pad 130′ of laser device 100′ may be used to indicate the desired setting for use of laser device 100′, including laser module 110 power level, pulse settings, treatment duration, etc.

Laser modules 110 may be positioned such that beams from each laser module 110 project to distinct points within a treatment region. Additionally, laser modules 110 may be positioned such that subsets of beams from laser modules 110 may impact the treatment region at substantially the same region, thereby providing one or more regions within the treatment area at which additional radiant energy is applied.

The hood-style laser device 100′ may further include one or more pins 115 disposed within housing 160, such as along the inner concave face 165, to provide stimulation and measurement capabilities within a treatment region, or to ensure laser device 100′ is properly positioned with respect to a treatment region.

While the above exemplary approach describes housing 160 of laser device 100 as having a concave inner face 165, it is to be understood that this is by way of example and not of limitation, and that housing 160 and inner face 165 may have any number of forms without parting from the scope of this disclosure.

FIG. 5C illustrates an embodiment of a device 100′ similar to that illustrated in FIG. 5A. In the illustrated embodiment, a plurality of laser modules 110 are disposed within the inner concave face 165 of housing 160. The laser modules 110 are mounted such that the outer surfaces of the modules are generally flush with, or recessed within, the inner concave face 165 of housing 160.

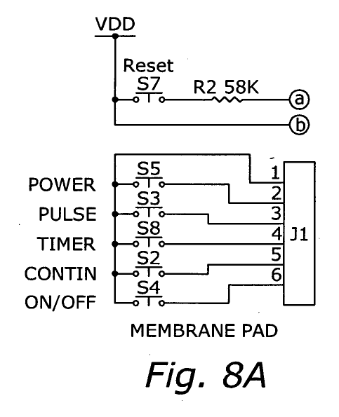

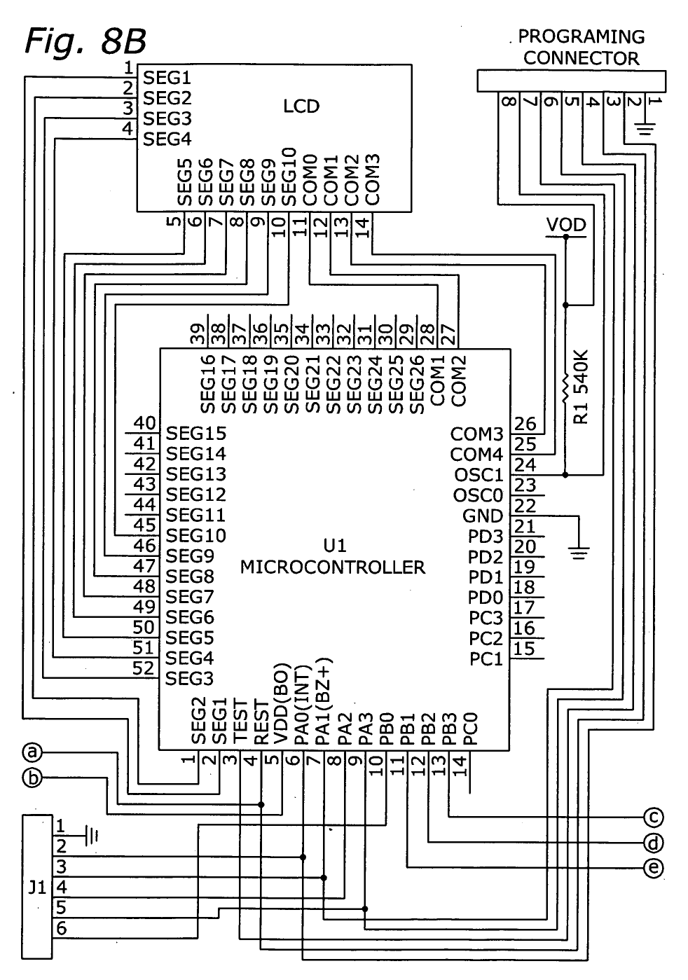

FIG. 6A illustrates an isometric view of an embodiment of a laser module 110. Laser module 110 may include mounting hardware 119 disposed along a proximate end thereof. Mounting hardware 119 may be configured to allow expedited positioning, focusing, insertion and/or removal of a laser module 110 within a device such as device 100′ of FIG. 5C.

FIG. 6B illustrates an exploded side view of the laser module 110 of FIG. 6A. The illustrated laser module 110 includes a laser diode 111 and a photo diode 112 mounted therein. The laser diode 111 is configured to project a beam through a lens 114 disposed at a proximate end of the module 110. The lens 114 may be, e.g., an expander lens configured to project a focused beam from laser diode 111 in a wider and/or more conical manner. Lens 114 may be disposed proximate mounting hardware 119. Laser module 110 may further include a control circuit (not shown) which may be configured to receive and/or provide signals or power to control output of laser energy from laser diode 111, and may be configured to receive information from photo diode 112.

The mounting hardware 119 in the exemplary embodiment includes double-sided adhesive 119c, e.g. double-sided tape, mounted to an outer face of a rubber washer 119b. A metal washer 119a may be disposed along an innermost face of rubber washer 119b. In an exemplary embodiment, metal washer 119a may be a concave washer, to better conform to contours of surrounding structure, e.g. a concave inner face 165 of housing 160. The mounting hardware 119 may allow for easy placement of a module 110 within a housing 160 of a device 100′, such as within concave inner face 165.

Laser module 110 may further include a connector portion 123 configured to allow quick connection and disconnection of a laser module 110.

While the above exemplary approach describes mounting hardware 119 of laser module 110 as including double-sided adhesive 119c as a mounting mechanism, it is to be understood that this is by way of example and not of limitation, and that mounting hardware 119 may have any number of forms without parting from the scope of this disclosure, such as a clasp, a magnet, a male or female end configured to clasp a corresponding female or male end of housing 160, etc. Further, the placement of the various elements which make up mounting hardware 119 are shown in a particular order, though this is by way of example and not of limitation. For example, the relative placement of the metal washer 119a, the rubber washer 119b, and the double-sided adhesive 119c and may be altered, to allow additional or alternative placement methods for laser module 110. Further, one or more of metal washer 119a, the rubber washer 119b, or double sided adhesive 119c may be omitted without parting from the scope of the present disclosure.

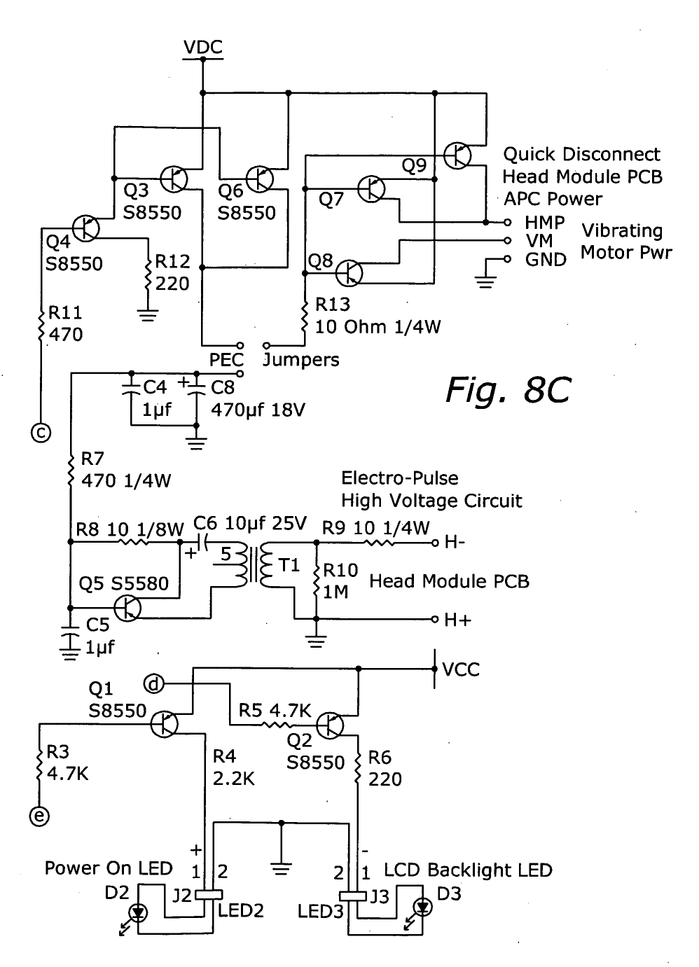

Functionally, a laser device 100 or 100′ operates in accordance with the flow chart of FIG. 7C. In step 200, the power on/off button on a control pad 130 is selected to turn on the laser device 100. In conjunction, a light emitting diode (LED) power “on” indicator on the upper housing (top) of the laser device 100 turns on, providing a visual verification that the laser device 100 is active. In addition, selecting the power on/off button powers the LCD display 125. Once power has been turned on in step 200, a predetermined delay is initiated at step 202 while the laser device 100 awaits a power level, pulse level, and time mode selection from the user. In one exemplary approach, the delay is approximately five seconds. Generally, the user has five power levels and five pulse levels from which to select. With respect to a time interval, the user may select either a continuous time mode for up to an hour, or one of several discrete time interval settings, such as five, ten or fifteen minute time interval settings. If the approximately five second delay expires without a selection being made, laser device 100 defaults to the last known selection. The order in which a power, pulse, or time selection is made is not germane to the functionality of laser device 100.

At step 204, the user selects a power level by selecting the “power” button on control pad 130. The power levels are pre-programmed into the device 100, such as in a microcontroller, and may be dependent on the number of laser modules 110 disposed in a specific laser device 100. In an exemplary device, however, power level one is programmed as a 20% duty cycle. Each additional power level represents an incremental increase with respect to power level one. In one exemplary approach, the incremental increase per power level is a 20% duty cycle. One of ordinary skill in the art understands that the incremental power levels, as well as the base power level, is a variable that may be pre-programmed and/or adjusted depending on the specifications of the application and the laser device 100.

At step 206, the user selects a pulse level by selecting the “pulse” button at the control pad 130. The pulse level in general represents the number of laser pulses applied to a treatment region every second, as well as the duration of each pulse. In an exemplary approach, pulse level one is programmed to two pulses per second. For each increase in pulse level, the number of pulse per second is increased by one. For example, pulse level two represents three pulses per second and pulse level three represents four pulses per second. Similar to the power level settings, one of ordinary skill in the art understands that the pulse level settings and the pulse duration are adjustable and programmable, and may be dependent on the specific laser device 100 application. In addition, the frequency of laser pulses may be modified by adjusting the pulse width duration or by modulating the pulse width. Pulsing the laser modules 110 advantageously reduces thermal runaway and power consumption of the laser modules 110, thereby extending battery life and service life of the laser device 100. In addition, by pulsing the laser modules 110 the cells, tissues, hair papilla, follicles and hair are allowed to rest and resonate, which may increase the effects of cellular stimulation, which may aid and promote cellular regeneration and proliferation, as well as hair growth.

At step 208, the user selects a timer function by selecting either the “timer” button or the “C.Mode” button on the control pad 130. By selecting the “timer” button, the user is able to select a five, ten or fifteen minute operation time interval. In other words, if the user selects five minutes, the laser device 100 will operate at the selected power and pulse level for five minutes. By selecting the “C.Mode” button, the user is able to customize the operation time interval subject to a maximum allotted treatment time, which may be sixty minutes.

After the user has selected all relevant settings, a delay timer may initiate at step 210. In one exemplary embodiment, the delay timer may be a five-second timer. At the end of the delay timer, the settings may initiate, to allow treatment to begin at step 212. Providing a delay timer may allow time for a user to position a device proximate a treatment area, and/or may serve as a safety mechanism to ensure a device 100 is properly positioned prior to activation of laser modules 110.

At step 214, following the conclusion of a selected time interval, the auto off function initiates. This function begins timing at the completion of the treatment session and automatically turns off laser device 100 after a period of inactivity. This inactive time period may vary and may be programmable. The inactivity timer may include a warning alarm to indicate to a user that the device 100 is to be powered off. The period may be adjusted depending on the specific laser device 100 needs and applications.

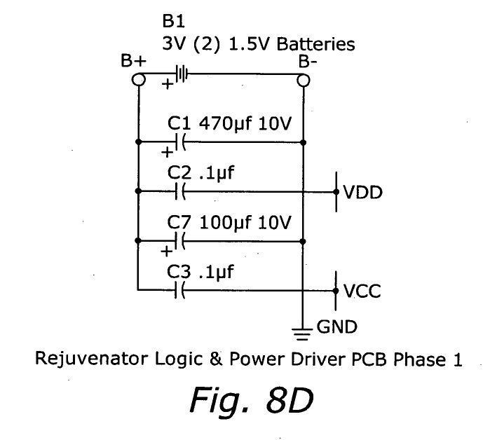

FIG. 8 illustrates an exemplary schematic of a laser device 100 and exemplary peripheral components.

While the present invention has been particularly shown and described with reference to the foregoing exemplary approaches, it should be understood by those skilled in the art that various alternatives to the exemplary approaches of the invention described herein may be employed in practicing the invention without departing from the spirit and scope of the invention as defined in the following claims. It is intended that the following claims define the scope of the invention and that the method and system within the scope of these claims and their equivalents be covered thereby. This description of the invention should be understood to include all novel and non-obvious combinations of elements described herein, and claims may be presented in this or a later application to any novel and non-obvious combination of these elements. The foregoing exemplary approach is illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

What is claimed is:

1. A device for providing laser energy to a treatment region, comprising:
a head assembly including a plurality of discrete laser sources configured to provide laser energy to the treatment region;
wherein at least a subset of the plurality of laser sources are positioned to provide laser energy to substantially the same portion of the treatment region.

2. The device of claim 1, wherein at least a portion of laser energy from a first laser source and at least a portion of laser energy from a second laser source act in a reinforcing manner.

3. The device of claim 2, wherein the at least a portion of laser energy from the first laser source and the at least a portion of laser energy from the second laser source overlap in a common area of contact within the treatment region.

4. The device of claim 1, further comprising a plurality of interchangeable head assemblies, at least a subset of laser sources associated with a first head assembly generating laser energy at a range of wavelengths dissimilar to the laser energy generated by at least a subset of laser sources associated with a second head assembly.

5. The device of claim 4, wherein the laser sources associated with a first head assembly generate laser energy at a range of wavelengths dissimilar to the laser energy generated by the laser sources associated with a second head assembly.

6. The device of claim 1, wherein at least a subset of the laser sources are selectively configured to generate laser energy in accordance with at least one predetermined pulsing interval.

7. The device of claim 6, wherein the at least one pulsing interval is selected to facilitate a resonance response within an area of contact within the treatment region.

8. The device of claim 6, wherein the at least one pulsing interval is selected to reduce current draw by the laser sources

9. The device of claim 6, wherein the at least one pulsing interval is provided by at least one laser source duty cycle, the duty cycle including an on cycle where the at least one laser source is powered and an off cycle where the at least one laser source is not powered.

10. The device of claim 9, wherein the on cycle is provided according to a sub-duty cycle, the sub-duty cycle including an on cycle and an off cycle.

11. The device of claim 1, further including a plurality of pins configured to selectively contact the treatment region, wherein the plurality of pins are offset with respect to the plurality of laser sources to prevent interference between the pins and the laser energy

12. The device of claim 11, wherein at least one of the plurality of pins includes an integrated sensor to selectively sense a treatment property of the treatment region.

13. The device of claim 12, wherein generation of laser energy is controlled in response to the sensed treatment property.

14. The device of claim 13, wherein laser energy is provided only upon detection of a sensed treatment property.

15. The device of claim 12, wherein the sensor senses at least one of pin deflection, temperature, electrical resistance, impedance, reactance, and current.

16. The device of claim 11, wherein at least one of the plurality of pins includes a varying treatment stimulus.

17. The device of claim 16, wherein the varying treatment stimulus is at least one of magnetic energy, vibration, a temperature offset from an ambient temperature, and a high-voltage electro-pulse.

18. The device of claim 16, wherein the at least one pin is operatively connected to at least one of a magnet, a motor, or a thermal regulator.

19. The device of claim 1, further comprising a data port configured to allow data transfer to and from the device.

20. A handheld self-contained laser device, comprising:
a laser control circuit and a plurality of discrete laser sources;
a first laser source positioned to provide laser energy to a first focal point;
a second laser source positioned to provide laser energy to a second focal point;
wherein at least a portion of the laser energy from the first laser source and at least a portion of the laser energy from the second laser source overlap;
the laser control circuit configured to cause at least a subset of the laser sources to generate laser energy in accordance with at least one predetermined pulsing interval; and
wherein the at least one pulsing interval is selected to reduce current draw by the laser sources, and to facilitate a resonance response within an area of contact within the treatment region.

21. The device of claim 20, further comprising a plurality of pins configured to selectively contact the body region, wherein the plurality of pins are offset with respect to the plurality of laser sources to prevent interference between the pins and the laser energy.

22. The device of claim 21, wherein at least one of the plurality of pins includes an integrated sensor to selectively sense a treatment property of the body region.

23. The device of claim 22, wherein generation of laser energy is controlled in response to the sensed property.

24. The device of claim 23, wherein laser energy is provided only upon detection of a sensed treatment property.

25. The device of claim 22, wherein the sensor senses at least one of pin deflection, temperature, electrical resistance, impedance, reactance, and current.

26. The device of claim 21, wherein at least one of the plurality of pins includes a varying treatment stimulus.

27. The device of claim 26, wherein the varying treatment stimulus is at least one of magnetic energy, vibration, a temperature offset from an ambient temperature, and a high-voltage electro-pulse.

28. The device of claim 26, wherein the at least one pin is operatively connected to at least one of a magnet, a motor, or a thermal regulator.

29. The device of claim 20, wherein the at least one pulsing interval includes at least a first portion where the plurality of laser sources are generally powered and at least a second portion where the plurality of laser sources are generally not powered.

30. The device of claim 29, wherein the plurality of lasers are selectively powered during the first portion according to at least one pulsing interval.

31. A method of providing laser energy to a treatment region comprising:
directing laser energy from a plurality of laser sources onto a treatment region from a plurality of directions;
pulsing at least a subset of the laser sources according to at least one pulsing interval;
directing laser energy from at least a subset of the plurality of laser sources onto substantially the same portion of the treatment region; and
reinforcing laser energy from a first direction by directing laser energy from a second direction, thereby increasing the energy provided to a treatment region.

32. The method of claim 31, further including selectively pulsing at least a subset of the laser sources, causing at least a portion of the treatment region to resonate and selectively reducing current draw by the laser sources.

33. The method of claim 32, further including directing laser energy along the shaft and into the root of a hair.

34. The method of claim 29, further including directing the laser energy through the shaft, into a hair follicle, and into the area surrounding the hair follicle.

35. The method of claim 31, wherein pulsing at least a subset of the laser sources according to at least one pulsing interval includes powering the at least a subset of laser sources during a first interval and removing power form the at least a subset of laser sources during a second interval.

36. The method of claim 35, wherein powering the at least a subset of laser sources during a first interval includes selectively powering a laser during at least a first sub-interval, and removing power from the at least a subset of laser sources during a second interval.
